(19) United States
(12) Patent Application Publication
(10) Pub. No.: US 2004/0009500 A1
(43) Pub. Date: Jan. 15, 2004

(54) Items with activated surface used for immobilisation of macromolecules and procedures for the production of such items

(75) Inventors: Rudiger Benters, Sande (DE); Christof M. Niemeyer, Bremen (DE); Dieter Wohrle, Bremen (DE)

Correspondence Address: Chimera Biotec GmbH, Ph.D. Hendrik Schroder, Emil Figge Str. 76 a, Dortmund, 44227, DE

(73) Assignee: chimera biotec GmbH, Bremen (DE)

(21) Appl. No.: 10/355,088

(22) Filed: Jan. 31, 2003

Related U.S. Application Data
(60) Provisional application No. 60/358,300, filed on Feb. 21, 2002.

(51) Int. Cl. C12Q 1/68; C12M 1/34; G01N 33/545

(52) U.S. Cl. 435/6; 435/287.2; 436/531

(57) ABSTRACT

Items described herein possess an activated surface for immobilisation of bioorganic macromolecules, i.e.:

a substrate with a surface

a dendrimeric scaffold attached to the substrate surface and

a number of primary residues bound to the dendrimeric scaffold for immobilisation of bioorganic macromolecules.

These items can be manufactured by using a procedure with the following steps:

provision of a substrate with a surface

linkage of the substrate surface with a dendrimeric scaffold and

supply of the dendrimeric scaffold with a number of primary residues for immobilisation of bioorganic macromolecules.

### PROVISIONAL APPLICATION FILED

Bremen, Aug. 13, 2002

| | |
|---|---|
| Inventors: | Dr. R. Benters |
| | Prof. Dr. G.M. Niemeyer |
| | Prof. Dr. D. Wöhrle |
| Assignee: | chimera biotec GmbH |
| | Kurfürstenallee 23a |
| | 28211 Bremen |
| | Germany |

### DESCRIPTION

### BACKGROUND OF THE INVENTION

The invention on hand covers items, especially sensors, with an activated surface for immobilisation of bioorganic macromolecules as well as the procedure of their production. The invented procedure covers particularly the production of activated surfaces of sensors for the highly effective covalent immobilisation of bioorganic macromolecules.

The detection of, for example bioorganic macromolecules using affinity reactions at biosensors requires substrate-surfaces which are coated with an reaction partner of the interacting molecule (solid-phase-method).

The durable fixation of this interacting molecule onto a sensor matrix (for example a substrate-surface) is enabled by a chemical modification to provide reactive residues for this immobilisation.

Those residues are usually reactive hydroxyl-, amino-, carboxyl-, acylhalogenid-, aldehyde-, isothiocyanate- or epoxy-residues. To covalently bind them to the above mentioned surface, they are coupled to a so-called spacer. This spacer-system is stable against oxidation and enables the linkage of the macromolecules onto the surface. Therefore the above mentioned technique of immobilisation seems to be very convincing as the sensor is regenerative and made for multiple use. This of course results in a reduction of cost.

Former strategies of immobilisation are based on linear linkers:

For example, surfaces made of silica oxide can be activated by coating with aminoalkyl-silans. Bioorganic macromolecules can then bind electrostatically. (Chrisey, L.; O'Ferrall, C. E.; Spargo, B. J.; Dulcey, C. S.; Calvert, J. M. Nucleic Acids Research 24/15 3040-3047 (1996)).

A second strategy consists of using gold-coated surfaces by coupling the macromolecules to a ω-functionalised alkylthiol-SAM (SAM=self-assembled-monolayer). (Bardea, A.; Dagan, A.; Willner, I. Anal. Chim. Acta 385, 33-43 (1998)).

If the concerning macromolecule contains a thiol-residue, a direct attachment to the goldlayer will happen by chemisorption, see also DE19807339 A1.

Glass surfaces can be functionalised by adding suitable silanes. Epoxyalkylsilane-activated glass surfaces are often employed to immobilise biomolecules with hydroxy- or amino-residues (U.S. Pat. No. 5,919,626). Macromolecules containing thiol-residues can be attached to Thiosilan-activated surfaces by disulfide-bridges (U.S. Pat. No. 5,837,860).

A further possibility of immobilisation is the use of avidin/streptavidin coated surfaces which show a high affinity for biotinylated substances (DE 3640412 Al; DE 19724787 Al).

However, the above mentioned techniques are improvable as these surfaces generally show less binding capacity and a lack of regenerative capability.

For example, the bonding of gold and sulphur is destroyed in the presence of oxygen; avidin-streptavidin surfaces are denatured while using thermal regeneration; and silylated glass surfaces show an instability when used in alkaline media with pH-values above 8 (Sandoval, J. E. et. al., Anal. Chem. 63, 2634-2641 (1991)). Moreover, silylated glass surfaces possess a low loading capacity which mostly results in an insufficient sensitivity of those sensors (Southern et al. Nature genetics supplement 21, 5-9 (1999)).

Meanwhile, there have been several projects with the aim to improve loading capacity and regenerative traits of surfaces, for example by using intermediate dextrane layers which are covalently (Löfas, S. et al. Pure AppI. Chem. 67, 829-834 (1995)) or chemi-sorptively (Johnsson, B.; Löfas, S.; Lindquist, G. Anal. Biochem. 198, 268-277 (1991)) bound to gold and show traits of hydrogels.

However, the immobilisation on these surfaces takes place inside the hydrogel matrix. The reactions in the interphases are heterogeneous and are limited by diffusion. This effects the access to the immobilised component. (Southern et al. Nature genetics supplement 21, 5-9 (1999)).

This problem also appears when surface-active micro particles (CPG, magnetic beads, Merrifield resin, etc.) are covalently attached to sensor surfaces to increase the surface (U.S. Pat. No. 5,900,481). These prepared surfaces are micro porous and are difficult to use for affinity reactions due to their limited diffusion.

Beier et al. describe a technique which inables the increase of potential binding sites for macromolecules on the sensor surface by in-situ building up branched linker-systems (Beier, M.; Hoheisel, J. D. Vol27/No9, 1970-1977 (1999)). However, this technique needs an preceding amination of the substrate (Silylation, RFPE=radio frequency plasma discharge in ammonia, etc.) and a repeated two-step synthesis, which takes app. 1-2 days. This is followed by an activation of the residues. Therefore eight or more reaction steps within several days are necessary to activate the surface which makes this technique time consuming.

### DETAILED DESCRIPTION OF THE INVENTION

In the view of the disadvantages of the known techniques the first task of the invention on hand was to create an item, i.e. a sensor, with an activated surface that has a high density of reactive residues. Moreover, it should have a high thermal and chemical stability towards the reaction steps used for regeneration.

The second task of the invention on hand was to create a technique which allows the establishment of a highly reactive surface within only few reaction steps.

The third task of the invention on hand was an instruction for a technique to produce an item (a sensor) with an immobilised bioorganic macromolecule on the surface.

The first task was solved by an item with an activated surface for immobilisation of bioorganic macromolecules, including:

A substrate with a surface

A dendrimeric scaffold attached to the substrate-surface

Several binding residues (for example NHS- or isothiocyanate-residues) bound to the dendrimeric scaffold and necessary for immobilisation of bioorganic molecules.

The item could be a (bio-) sensor or an component of such, for example a DNA-microarray, an protein-array of antibodies, receptors and/or enzymes, an array of peptides or peptoides, or a compound of low molecular weight such as pharmacophores or other active substances.

The dendrimeric scaffold can contain a variety of identical or differing residues, depending on the requirements (e.g. NHS-esters, isothiocyanates, strands of nucleic acids and suchlike). Sensors should usually be capable to detect different kinds of analytes. Therefore it seems useful to offer corresponding primary residues to those different analytes.

The dendrimeric scaffold is attached to the substrate-surface by a linking unit. This linking unit is formed by a reaction of an initial residue on the substrate surface with a complementary functional residue on the dendrimeric scaffold (synthesis of this item, see below). If the initial residue on the surface originally carries a carboxy function and the complementary residue of the dendrimeric scaffold an amino function, then the linking unit is an amide. Further examples for initial and complementary residues will be explained in the chapter for the inventive production techniques. The expert, knowing the educt-residues, will be aware of the structure of this linking unit.

The dendrimeric scaffold can include the following dendrimeric components which can also be linked to each other within the dendrimeric scaffold: Starburst dendrimers and their chemically and biochemically modified derivates; metallodendrimers, carbosilane dendrimers, polysilane dendimers, glycosyl-containing dendrimers, saccharide- and oligosaccharide dendrimers and their derivates, peptide- and oligopeptide dendrimers and their derivates as well as nucleotide- and oligonucleotide dendrimers and their derivates.

The dendrimeric scaffold contains primary residues for immobilisation of bioorganic macromolecules and other substances. These residues derive from a reaction between a functional residue in the dendrimeric scaffold and a homo- or hetero functional linker substance (or linker molecule) (synthesis of this item, see below).

If the functional residue of the dendrimeric scaffold carries an amino-function and reacts with the homobifunctional linker substance phenylen-1,4-diisothiocyanat, then the primary residue of the dendrimeric scaffold results in an isothiocyanate-residue, if the functional residue of the dendrimer is an amino group and reacts with an bis-epoxyd linker molecule then the primary residue is an epoxyd-residue.

Further examples for linker substances will be mentioned in the chapter for the inventive production techniques. The expert, knowing the educt-residues, will be aware of the structure of the primary residue. It has a single reactive function when using bifunctional linker molecules.

The dendrimeric scaffold also contains dendrimeric basic units which are attached to the substrate surface and can also be linked with each other using bi-functional linker substances (linker molecules). The advantages resulting from this technique see below.

The second task will be solved by a technique of the production of an item with an activated surface for immobilisation of bioorganic macromolecules using the following steps:

Provision of a substrate with a surface

Linkage of the substrate surface with dendrimeric units (esp. basic units of the dendrimeric scaffold)

Supply of the dendrimeric scaffold with several primary residues for immobilisation of bioorganic macromolecules.

The resulting item then shows the properties of the above described item.

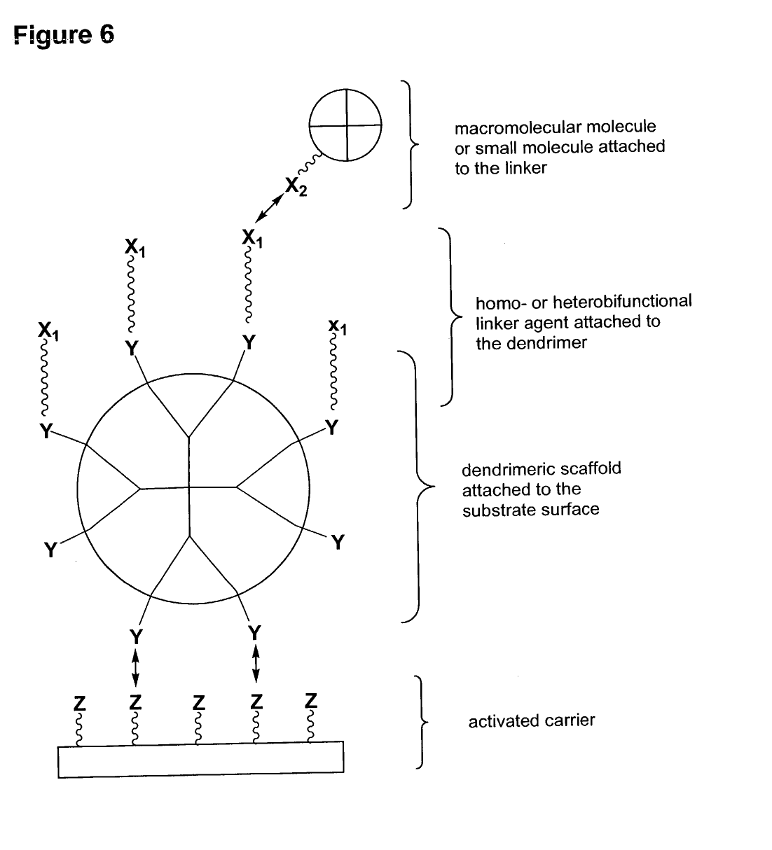

The substrate surface will usually be modified by a reactive initial residue, which means the initial residue will first be attached to the (unmodified) substrate surface. In the following step the substrate surface (now modified) will be linked to the dendrimeric scaffold (further modification). The modification of the substrate surface can be done by using standard techniques; see FIG. 6.

The reactive and surface-bound initial residue can be a member of the following chemically reactive residues: Hydroxyl-, amino-, carboxyl-, acylhalogenid-, ester-, aldehyde-, epoxy- or thiol-residues.

It can also be a member of the following biologically or chemically reactive residues: disulfides, metallochelats, nucleotide- or oligonucleotides, peptides or haptens, for example biotin-, digoxigenin-, dinitrophenyl-residues or similar residues.

The (unmodified) substrate surface which binds the reactive initial residue is preferably a member of the following group: carrier material based on metals, semi-metals, semi-conductors, metal- and non-metal-oxides, glass, plastics, organic and inorganic polymers, organic and inorganic films and gels, especially as a coating for one of the above mentioned materials.

The dendrimeric scaffold which is usually linked to a modified substrate surface by a reactive initial residue, can contain several identical or different functional residues per dendrimeric basic unit. They mostly belong to aminoalkyl-, hydroxyalkyl- or carboxyalkyl-residues. They are bound to the peripheral region of the dendrimeric basic unit and can be transferred into a primary residue by using a linker substance.

The dendrimeric basic units is preferably a member of the following group: Starburst-dendrimers and their chemically and biochemically modified derivates, metallodendrimers, dendrimers containing glycosyl-, saccharide and oligosaccharide dendrimers and their derivates, peptide and oligopeptide dendrimers and their derivates as well as nucleotide and oligonucleotide dendrimers and their derivates.

Dendrimeric structures, corresponding the dendrimeric basic units in the invented item, are usually synthesised prior to the invented procedure. This synthesis is performed in a standard way, the resulting dendrimers are then prepared for the invented procedure.

The in-situ construction of these dendrimers can be performed by the following processes:

(a) a parallel process of covalent and non-covalent interactions, such as self-organisation based on hydrogen-bonds, van-der-waals, coulomb or metal-ligand interactions

(b) a successive process of covalent and non-covalent interactions, such as self-organisation based on hydrogen-bonds, coulomb or metal-ligand interactions.

The dendrimeric basic units (i.e. educt-dendrimers and the resulting dendrimeric scaffold) are provided with a number of residues for immobilisation of bioorganic macromolecules. This is done by a reaction between the functional residue of the dendrimeric basic units with a homo- or heterobifunctional linker substance (linker molecule).

It is possible to provide the dendrimeric basic units with primary residues before or after a further linkage of the dendrimeric basic units to the substrate-surface.

Homobifunctional linker substances can be a member of the following group:

Photochemically, chemically, biochemically or biologically active compounds such as dicarbon acids and their anhydrides, disuccinimidylglutarat, phenylendiisocyanates, bis-epoxyethyleneglycoles bis-[β-(4-azidosalicylamido)ethyl] disulfides, 1,4-bis-maleimidoalkanes, 1,6-hexan-bis-vinylsulfone.

Heterobifunctional linker substances can be a member of the following group:

Photochemically, chemically, biochemically or biologically active compounds such as 3-[(2-aminoethyl) dithio]propion acid, N-[β-maleimidoacteoxy]succinimide, N-[κ-malimidoundecan acid ]hydrozide, succinimidyl-6-[3-(2-pyridyldithio)-propionamido] hexanoate, N-succinimidyl-iodoacetate.

Knowing the functional residues of the dendrimeric basic units as well as the corresponding bifunctional linker substances, an expert can precisely predict the functionalities of the dendrimeric scaffold of the item.

Due to the physicochemical stability a certain technique of production seems to be preferable (therefore also the resulting items). This immobilization technique consists of a cross-linkage of the surface-bound dendrimeric basic units using homobifunctional linkersubstances (except carbon acid anhydrides). It results in a thin, polymeric layer of dendrimeric units. Those linker substances are preferably but not necessarily the same as the ones used for synthesis of the functional residues. It is referred to the above mentioned preferred linker substances.

Due to a high immobilisation capacity a certain technique of production seems to be preferable (therefore also the resulting items). This procedure consists of a synthesis of the primary residue by using heterobifunctional linker substances or carbon acid anhydrides. This procedure converts every functional residue of a dendrimeric basic unit into an active residue. It is referred to the above mentioned preferred linker substances.

The selection of dendrimers (dendrimeric basic units) and linker substances is done depending on the immobilised substances and on the capability of cross-linkage between dendrimeric units.

According to the preferred technique of production a macromolecular surface is built up by cross-linking the dendrimers attached to the surface. These resulting surfaces show two advantages compared to common linear linkage systems. (Covalent) cross-linkages of immobilised dendrimers give a higher physicochemical stability to the surfaces. It also allows a loss-free regeneration of surfaces which are loaded with bioorganic macromolecules (for example sensor-surfaces).

Furthermore the use of poly-functionalised dendrimers drastically increases immobilisation efficiency. Due to the chemical nature of dendrimers the linker system shows a high flexibility in combination with a high sensitivity for heterogeneous affinity reactions. Examples are hybridisations of nucleic acids and of antibody-antigen, of protein-protein, of protein-ligand or of receptor-substrate-interactions.

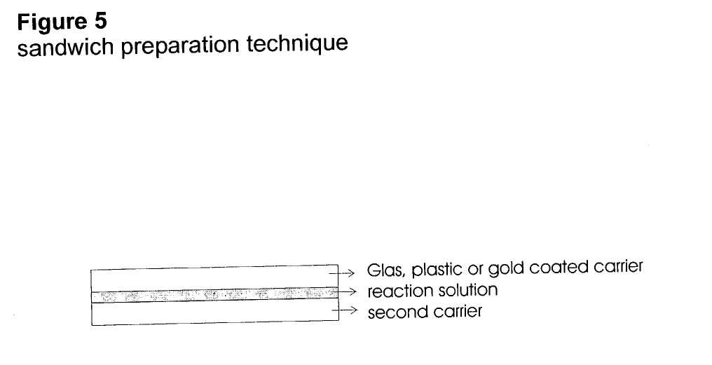

A most preferable procedure for the construction of the dendrimeric scaffold and therefore an essential step for the production of the invented item is a so-called sandwich-preparation, used for example for the construction of an amino-dendrimeric sensor surface. As shown in FIG. 5, two activated substrates such as glass slides measuring 3×8 cm (different sizes can also be used) can be assembled in pairs to enable the immobilisation of dendrimers in the intermediate space between those two substrates. These two substrates were treated beforehand by aminosylation and activated with disuccinimidylglutarate. When using glass slides 100 μl of a 10% solution of (amino-) dendrimers is applied onto one of the slides. By laying the second slide on the first slide the drop of (amino-) dendrimer-solution will then be spread to form a thin layer of (amino-) dendrimer-solution between those two substrates. This procedure clearly shows two advantages; first the thin layer improves the reaction speed and also the homogeneity of the surface reaction. Secondly only little amounts of the expensive reaction solutions are necessary. This sandwich preparation can also be used for synthesis of the primary residue. This is done by applying a saturated solution of the linker substance, such as glutaracid anhydride or 1,4-phenylendiisocyanat, onto substrates which are coated with dendrimeric basic units.

An advantageous approach of the invented procedure is to modify large planar substrates with dendrimer layers using the above described steps. Those substrates correspond to a multiples of a chip surface desired.

A typical approach is the use of 100×100 cm glass slides, but also slides of other materials or even larger measurements. These slides are cleaved into formats of the desired chips using well-known physical, mechanical or chemical procedures. The glass slide (or any other substrate) can then be divided into the format of the chips. These steps are usually followed by a chemical modification as mentioned before (and below), e.g. cleaning, silylating and activating steps, for example using baths. The application of the dendrimers is done following the sandwich-technique, as described earlier. The final activation of the dendrimers by using homo- or heterobiofunctional linkers is preferably performed using the sandwich or bath-treatment.

Afterwards the substrate slides are divided into their final size.

Alternatively dendrimer-coated slides can be divided and then, when required, be activated by bifunctional linkers

The advantage of this procedure (as well as their mentioned alternatives) is the scale-up of the chip-production and to gain a higher number of items of activated chip-surfaces by using the advantageous sandwich-technique.

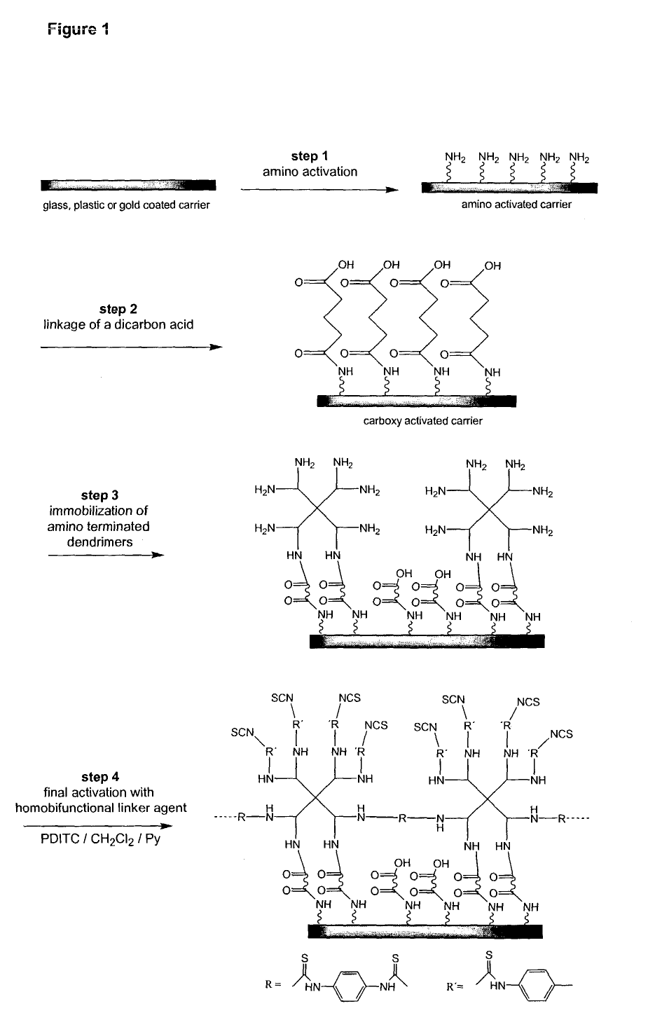

Due to the invented technique it is possible to attach a cross-linked dendrimeric scaffold with free residues to a substrate surface (FIG. 1).

This macromolecular intermediate layer

(a) increases the number of potential binding sites for immobilisation of bioorganic macromolecules or other substances

(b) improves the physicochemical stability of the surface and

(c) improves the regenerative power of the substrate surfaces (carrier) which are modified with bioorganic macromolecules.

The invention on hand also includes items that are obtained from the invented procedure (including the above mentioned design of techniques).

The invented items include functionalised solid phases und can be used as sensor components, reactor components and components with electrical, electronical or optical functions. The items functionalised by bioorganic macromolecules can, besides the mentioned biosensors (DNA-and protein-arrays), also be employed as solid phases in enzyme reactors in which a high physicochemical resistance and high loading densities are essential.

Because of the possibility to functionalize DNA-microarrays of high integration density with inorganic colloids and semiconductors (Niemeyer, Curr. Opin. Chem. Biol., 4, 609 (2000)), these robust surfaces can also be used for optically active devices, such as high-resolution displays.

The third task is solved by a technique of the production of an item with an immobilised molecule (macro- or other) on the surface, described in the following step:

contact of a macromolecule containing at least a second residue

(a) with the invented item

(b) with an item which was produced using the invented procedure and contains an activated surface

under conditions in which at least one first residue of the item reacts with the second residue of the macromolecule to form a linkage between the macromolecule and the item.

Apart from macromolecules also other substances which are attached to accordingly prepared (a or b) invented items, can be immobilised, like macromolecular colloids and nano-particles or compounds of low molecular weight such as pharmacological substances, hormones, antigens and other active substances.

The invention also includes items that are molecules (macro or other) linked to a dendrimeric scaffold. This (macro-)molecule can be a member of the following group: antibodies, especially IgG, IgM, IgA, enzymes; receptors; membrane proteins, glycoproteins; carbohydrates; nucleic acids; for example DNA, RNA, peptide nucleic acids (PNA), pyranosyl-ribonucleic acid (pRNA).

The term “other substances” does include organic- or inorganic-chemical residues with specific or potentially effective pharmacological, catalytical (for example photocatalytical active substances such as porphyrins and their derivates or catalysts for a stereo-selective re-structuring of organic or inorganic substrates), optical or electrical traits (for example fluorescent, electro-luminescent compounds or electrically conductive polymers (polyanilin, polypyrrol)).

Libraries of substances which are accessible by a combinatory solid phase synthesis can also be immobilised on the item. This is done to screen for functionality of the attached substances, for example their inhibition of biological enzymes or receptors.

The principles of the invention on hand is, among others, the surprising discovery that glass surfaces, produced by the invented technique and functionalised with nucleic acids show a drastically improved sensitivity for the detection of complementary nucleic acids. There is furthermore an increased physicochemical stability which makes treatment for regeneration of the nucleic acid-modified carriers possible. This treatment with alkaline washing solutions can be repeated several times without an activity loss of the surface. It was discovered that items which were made using the invented procedure had a significantly improved physicochemical stability and therefore show a loss-free regeneration of the carriers modified with bioorganic macromolecules. Moreover, the chemical nature of dendrimeric units causes a high flexibility of the linker system for the binding of the bioorganic macromolecules. This results in a higher efficiency of the heterogeneous affinity reaction compared to linear linker systems.

As mentioned before it is possible to attach a cross-linked dendrimeric scaffold with free residues to a substrate surface to create a macromolecular intermediate layer (FIG. 1). This macromolecular layer can firstly be attached by treatment of either amino-, epoxy-, or carboxyl-modified glass surfaces (as substrate surface) with a dendrimeric macromolecule; and secondly with a homo- or heterobifunctional linker reagent. The number of binding sites for an immobilisation of bioorganic macromolecules (for example nucleic acids) is increased by binding dendrimeric components to a modified glass surface. The treatment with a homobifunctional linker reagent mostly results in

(a) an activation of the dendrimeric basic units (attached to the substrate surface) for immobilisation of bioorganic macromolecules, i.e. a supply with free residues and

(b) a covalent cross-linkage of the dendrimeric basic units.

Bioorganic macromolecules can be immobilised very stable on the surface of an item manufactured in that way.

The invention will be further explained below referring to examples and the enclosed figures.

### EXAMPLES

### Example 1

Procedure for the production of a glass carrier with a surface-attached cross-linked dendrimeric scaffold with free isothiocyanate-residues or epoxy-residues respectively; and immobilisation of a bioorganic macromolecule by coupling to residues

1.1. Aminosylation of a Glass Surface (Surface Activation)

A glass slide with a silica oxide surface was provided. The glass surface was thoroughly cleaned. The glass surface was then silylated (FIG. 1; step1), using a well-known procedure (Maskos, U.; Southern E. M.; Nucleic Acids Res., 20(7), 1679-1684 (1992)) with 3-aminopropyltrithoxysilane in ethanol/water (95:5). Other methods for silylation are also usable.

1.2. Carboxy-Functionalising of the Surface

The aminosylated glass surface (see 1.1) was then carboxy-functionalised using a 10 mM solution of disuccinimidylglutarate in CH₂Cl₂/n-ethyldiisopropylamine (100:1) and incubated 2 hrs in an argon-atmosphere at room temperature (FIG. 1; step2). The surface was then thoroughly cleaned with CH₂Cl₂.

By this procedure a carboxy-residue was attached to the surface which could afterwards be used as initial residue.

1.3. Linkage of the Amino-Terminated Dendrimers to a Surface (Amino-Dendrimer Immobilisation)

The surface was covered with a 10% solution of an amino-dendrimer known as Starburst (PAMAM) (Aldrich Chem. Co, also: Yamakawa, Y et al. J. of Polymer Science: Part A 37 3638-3645 (1999)). The solution was made in methanol (FIG. 1; step3). This step can easily be performed with the sandwich-technique as described above. After a reaction time of 30 min (reaction of carboxy-residues and amino-residues) the excess dendrimer was removed by a washing step. The dendrimer-containing (glass slide) surface was then dried in a nitrogen-atmosphere.

1.4. Conversion of Linker Substance (Production of Free Residues and Cross-Linkage of Dendrimeric Basic Units)

As described in 1.3. the glass slide was now provided with dendrimers. To obtain isothiocyanate activated surfaces the dendrimer coated glass slide is transferred into a 20 mM solution of 1,4-phenylen-diisocyanate, diluted in CH₂Cl2/Pyridin (100:1) (FIG. 1; step 4). After a reaction time of 30 min the slide was washed thoroughly in CH₂Cl₂. To obtain epoxy functionalized surfaces the dendrimer coated slide is treated with an bis-epoxyethylenglycole for approximatly 2 h. Afterwards the slide was washed thoroughly with acetone. Other homobifunctional linker molecules can be applied as well. The resulting glass slide with dendrimer-surface was then ready for immobilisation of bioorganic macromolecules or could be stored in argon for further purposes.

1.5. Linkage (Immobilisation) of Bioorganic Macromolecules—General Instruction

The surface as prepared in 1.4. was covered with a few drops of a watery solution containing a bioorganic macromolecule for immobilisation. This bioorganic macromolecule should have a concentration ranging from 1-100 μM. The bioorganic component could be a 5′-amino-modified oligonucleotide as purchasable from a number of commercial suppliers.

The covered surfaces were incubated for several hours in a humidity chamber. The optimal incubation time depended on the kind and concentration of the component to be immobilised. To avoid unspecific bindings, residues which had not reacted during incubation time were inactivated. Therefore the glass slide was transferred into a 6-aminohexanol-solution (100 mM in demethylformamid (DMF)) to inactivate active isothiocyanate-residues or epoxy-residues respectively (see 1.4. above). Afterwards the glass slide surface was treated with solutions containing detergents, for example sodium-dodecylsulfate, to remove non-covalently bound bioorganic macromolecules. It was then washed several time in distilled water and dried in a stream of nitrogen. Loaded sensors were stored at −20° C. for further purposes.

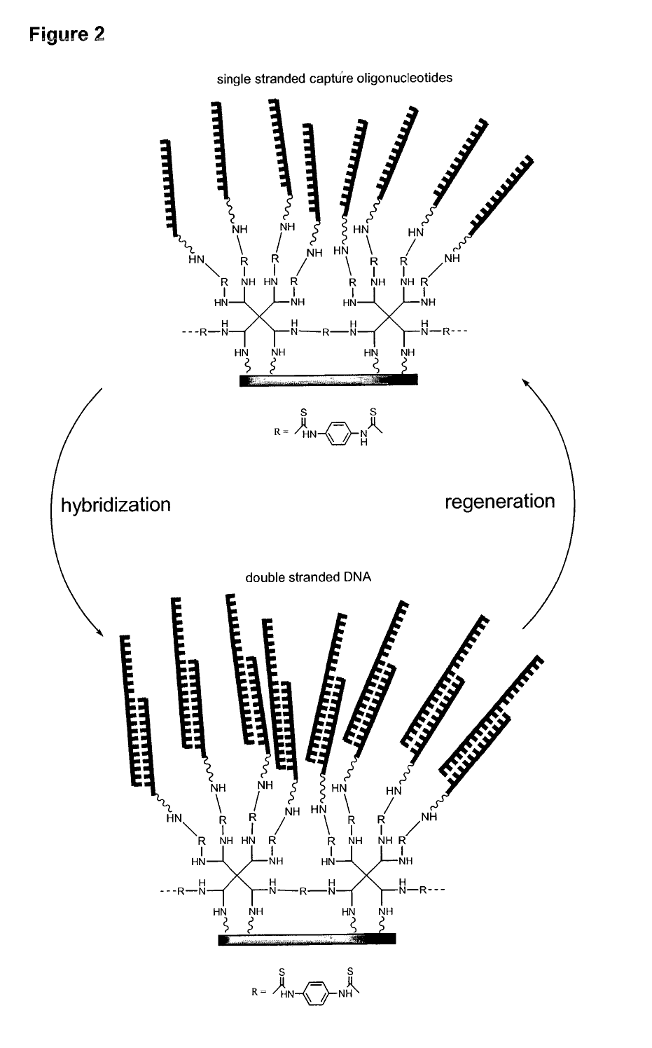

Dendrimer-based, isothiocyanate- and epoxy-functionalised surfaces that are loaded with for example amino-functionalised oligonucleotides, can be of high interest as sensor surfaces in DNA-chip-technology (Niemeyer, C. M.; Blohm, D. Angew. Chem. 111/19 3039-3043 (1999)). See also FIG. 2 and the corresponding explanations.

### Example 2

Variation 1: Modification of Glass Side Surfaces Using Carboxy-Terminated Dendrimers

Amination of a glass-based carrier was explained in example 1 (1.1.).

To establish a carboxy-dendrimeric surface a carboxy-terminated dendrimer (trade name Starburst (PAMAM) dendrimer; (Aldrich Chem.Co) was estered in the presence of dicylohexylcarbodiimid/N-hydroxysuccinimid using the well-known techniques (Johnsson, B.; Löfas, S.; Lindquist, G. Anal. Biochem. 198 268-277 (1991)). Activated dendrimer was transferred onto the aminosilylated surface, in the presence of DMF. After a reaction time of 30 min the excess dendrimer was removed using DMF. The surfaces could then be used for immobilisation of bioorganic macromolecules, as described in example 1. Alternatively it is possible to store them in argon.

### Example 3

Variation 2: Establishment of Macromolecular Sensor Surfaces Based on Amino-Reactive Silane Surfaces

The cleaning of glass surfaces is done as described in example 1, chapter 1.1.

Silylation using hydrolysis-stable silanes, such as 3-carboxypropyltrialkoxysilan, is performed as mentioned in example 1, chapter 1.1. Silylation with hydrolysis-sensitive silanes is preferably performed under dry conditions, as described elsewhere (Southern, E.M. et al.; Nucleic Acids Res., 22(8), 1368-1373 (1994)); other procedures are also conceivable. Such silanes could be 3-glycidoxypyropyltrimethoxysilane, 2-(3,4-epoxycyclohexyl)-ethyltriethoxysilane, 3-iodopropyltrimethoxysilane 3-isothiocyanatotrialkoxysilan and the corresponding trihalogensilanes. Silane surfaces terminated with epoxy-, isothiocyanato- and iodo-residues are used directly for linkage; whereas silane surfaces terminated with carboxy-residues have to be activated by a reaction with DCC/NHS, as shown in example 7 below, chapter 7.2. A further reaction of the dendrimeric surface with homo- and heterobifunctional spacers to generate the primary residue and a linkage of macromolecules, is performed as shown in example 1, chapters 1.4.-1.5.; as well as example 7, chapter 7.4.

### Example 4

Variation 3: Use of Carriers Made of Synthetic Material (Plastic)

Surfaces of plastic such as polyethylene, polypropylene, polystyrol, polycarbonate, polyacryinitril or their co-polymers were aminated by radio frequency plasma discharge in an ammonia-atmosphere according to procedures as described for example by Hartig, A. et al. Advances in Colloid and Interface Science 52, 65-78 (1994)

The establishment of dendrimer-based surfaces was performed as described in example 1, 2 and 7.

### Example 5

Variation 4: Use of Gold-Coated Carrier Materials

Gold-layers were applied on different carriers using common methods and aminated by well-known procedures, for example by producing a amino-terminated SAM on a gold-layer, as described by Glodde, M.; Hartwig, A.; Hennemann, O. -D.; Stohrer, W. -D. Intern. J. of Adhesion & Adhesivs 18 359-364 (1998).

The production of dendrimer-based surfaces and the attachment of bioorganic macromolecules was performed as described in example 1-3.

### Example 6

Variation 5: Use of Surfaces Based on Silica and Other Metals and Semiconductor Carrier Materials

Metal and semiconductor surfaces were activated using well-known procedures, for example by silylation with amino-, epoxy-, carboxy or thiosilanes (Chrisey, L.; O'Ferrall, C. E.; Spargo, B. J.; Dulcey, C. S.; Calvert, J. M. Nucleic Acids Research 24/15 3040-3047 (1996)) and (Bhatia, S. K. et al. Anal. Biochem. 178, 408-413 (1989)) or by hydrosilylation with ω-undecencarboacid (Sieval, A. B. et al.; Langmuir 14(7)1759-1768 (1998).

The concerning activated surfaces were then modified with a dendrimeric component, as shown in examples 1-3.

Bioorganic macromolecules were immobilised as described in example 1, chapter 1.5.

### Example 7

Variation 6: Procedure for Establishment of a Glass Carrier with a Surface-Bound, Unlinked Dendrimeric Scaffold with Free NHS-Ester-Residues.

7.1. Aminosilylation of a Glass Surface (Surface Activation):

Silylation was performed as in example 1, chapter 1.1.

7.2. Carboxy-Functionalising of the Surface

The aminosilylated glass surface was prepared as in 1.1. and then carboxy-functionalised with a saturated solution of glutaric acid anhydride in DMF, incubating 4 hrs at room temperature in an argon-atmosphere. The surface was then thoroughly washed with DMF and water. In the following step carboxy-residues were activated using dicyclohexylcarbodiimide (DCC) and N-hydroxysuccinimide (NHS). This was done by coating the surface with a solution of 1 M DCC and 1 M NHS in DMF. After a reaction time of 4 hrs the carrier was thoroughly washed with DMF and acetone.

The resulting NHS-ester-activated carboxy-residue was then usable as initial residue.

7.3. Linkage the Surface to an Amino-Terminated Dendrimer (Amino-Dendrimer-Immobilisation)

The linkage of the surface with the dendrimeric basic unit was performed as in example 1, chapter 1.3.

7.4. Reaction with a Linker Substance (Generation of Free Residues)

The glass surface was supplied with dendrimeric basic units as described in 1.3. and was then transferred into a saturated solution of glutaric acid anhydride in DMF. After a reaction time of 4 hrs a washing step with DMF and water was done. Afterwards the free carboxy-residues were activated with dicyclohexylcarbodiimid (DCC) and N-hydroxysuccinimide (NHS). This was done by coating the surface with a solution of 1 M DCC and 1 M NHS in DMF. After a reaction time of 4 hrs the carrier was thoroughly washed with DMF and acetone. The glass carrier with a dendrimer-surface was then ready to use for immobilisation of bioorganic macromolecules. It could also be stored in an argon atmosphere for further use.

Example 1 to 7 are referring to FIGS. 1 and 2 which will be further explained as follows:

FIG. 1: surface modification for production of a dendrimer-based macromolecular sensor surface.

FIG. 2: Linkage of biomolecules to the surface as shown in FIG. 1

FIG. 1 shows a surface modification of a glass carrier, or of plastic or a gold-coated carrier (as substrate); see also examples 1, 4, 5 and 7.

Step 1 shows an amino-activation of the surface by silylation, RFPD or ω-aminoalkylthiol-SAM, see also example 1, 1.1.

Step 2 shows the linkage of a dicarbon acid to a carboxylated surface, see also example 1, 1.2.

In step 3 a poly-functionalised amino-dendrimer is attached to this surface. Usually a dendrimer of the 4^{th} generation containing 64 amino-residues is used, see also example 1, 1.3.

The final activation of residues and a cross-linkage of the attached dendrimers is shown in step 4. For clarity reasons intramolecular cross-linkages of final amino-residues are not shown, see also example 1, 1.4.

FIG. 2 shows a dendrimer-based, isothiocyanate-functionalised surface loaded with bioorganic macromolecules. These macromolecules are amino-functionalised oligonucleotides. Such oligonucleotides are of special interest on sensor-surfaces used in DNA-chip-technology (Niemeyer, C. M.; Blohm, D. Angew. Chem. 111/19 3039-3043 (1999)). The attachment of these oligonucleotides is performed as described in example 1, 1.5. A regeneration can be done in alkaline environment (for example under following conditions: Use of 50 mM NaOH, room temperature, 2×3 min)

### Example 8

comparative evaluation of homogeneity and loading density on DNA-arrays which were produced (a) with a conventional linear linker and (b) with the invented surface modification.

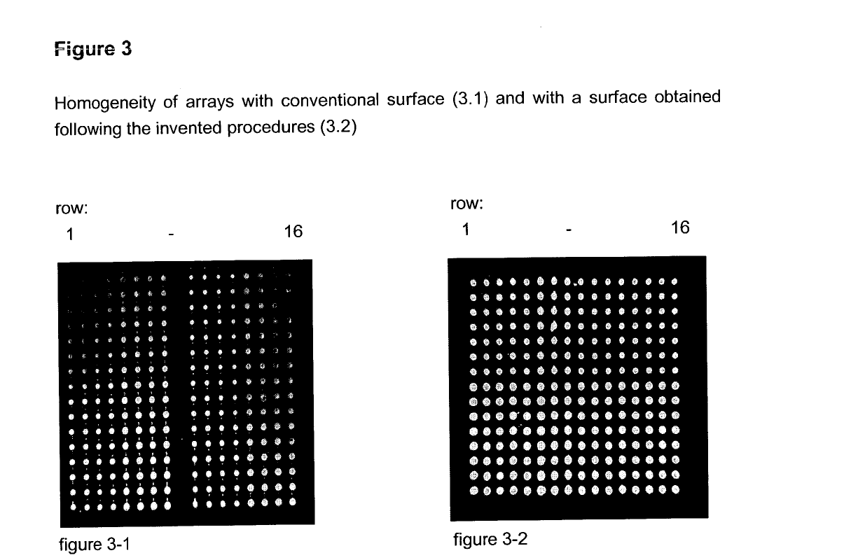

The examination is further explained in FIG. 3 (also FIG. 3-1 and FIG. 3-2).

FIG. 3-1 shows a conventional DNA-array carrying 5′-amino-modified capture oligonucleotides which are attached to the surface by a conventional linear linker.

Activation of the surface is performed using the following procedure:

After silylation with 3-aminopropyltriethoxysilane (see example 1; 1.1.) 1,4-phenylendiisothiocyanate was bound. Single spots were applied onto the conventional surface using a Piezo ceramic pipette to form an oligonucleotide-array. Spots in a vertical row were applied in the same volume of capture oligonucleotide solution:

rows 1-4, 9-12=2 nl; 5-8, 13-16=4 nl. Concentration of the spotted oligomer solution was 10 μmol/l diluted in H₂O_{bidest}.

In comparison to this, FIG. 3-2 shows a DNA-array carrying the invented sensor surface.

The method of modification is described in example 1.

The same capture oligonucleotide was immobilised as in FIG. 3-1.

To establish the invented array the volume of spots in rows 1-16 was varied:

7 upper spots: 2 nl; 8 lower spots: 4 nl. Concentration of the spotted oligomer solution was 10 μmol/l diluted in H₂O_{bidest}.

Hybridisation and comparative array-evaluation:

In both cases hybridisation was incubated 1 h at room temperature using a 1 nM solution of 3′-Cy5-labelled, complementary nucleic acid. Evaluation of arrays was done by fluorescent stimulation and visualisation with a common CCD-camera.

The array (FIG. 3.1) established using common procedures, i.e. activation by isothiocyanate of an amino-silylated glass surface, shows an inhomogeneous signal intensities within row 1-16 although a similar intensity had been expected due to identical conditions. This effect derives from an inhomogeneous surface modification. This is hardly avoidable when not using the invented procedure for surface modifications for arrays. These effects would definitely impede an evaluation of those arrays.

However, arrays whose surfaces were activated following the invented procedures (FIG. 3-2) show almost identical signal intensities in vertical rows. The homogeneity of surface modification is therefore clearly improved by the invented procedure.

Different loading densities using capture nucleotides can be explained by different exposure times used for the shots. For shot (FIG.) 3-1 an exposure time of 120 sec was necessary to detect signals; for shot (FIG.) 3-2 only 10 sec were needed.

### Example 9

Comparative evaluation of a regeneration stability

of an DNA-array with conventional linear linker and

of an array established by the invented surface modification

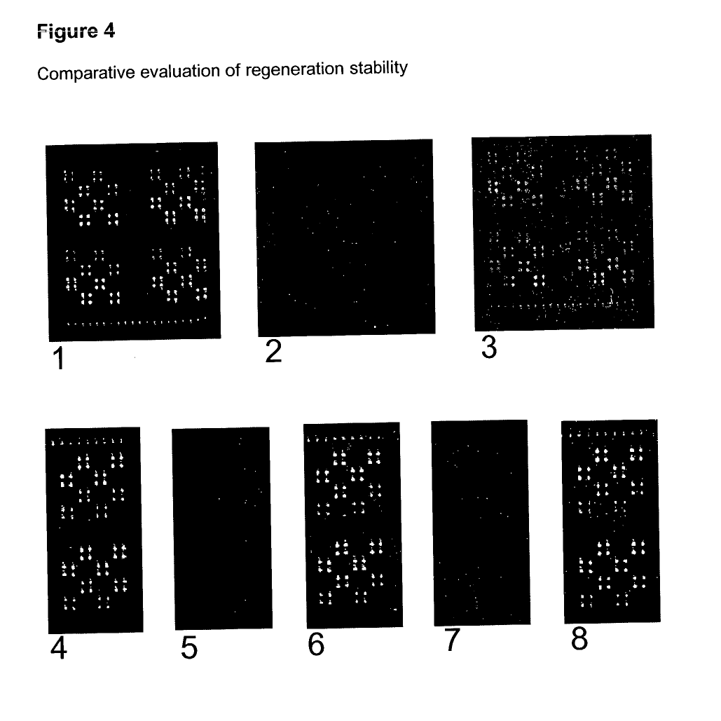

The evaluation is further explained in FIG. 4 (including FIGS. 4-1 to 4.8).

DNA-array shown in FIGS. 4-1 to 4-3 contains a conventional linear linker, whereas the array shown in FIGS. 4-4 to 4-8 posses an invented sensor surface. Surface modification and hybridisation of arrays was performed as described in example 7. Quarters of different volumes (1,4-5,6 nl) were spotted onto the surface. A regeneration step was done using 50 mM NaOH, 2×3 min at room temperature.

FIG. 4-1 shows the array with linear linker after a first hybridisation, FIG. 4-2 shows the same array after regeneration and FIG. 4-3 also shows it after a second hybridisation. A decreased signal intensity is clearly visible after the first regeneration step. One reason is the detachment of the capture nucleotides during the regeneration process due to the inherent instability of the conventional linker.

FIGS. 4-4 to 4-8 show the improved regeneration traits due to the invented process. Even after repeated regeneration steps a decrease in signal intensity can not be detected.

Further figures:

FIG. 5 is a schematic representation of the preferred sandwich-preparation-technique, see above.

FIG. 6 is a schematic representation of a preferred invented procedure for the production of an item with an activated surface for immobilisation of macromolecules and other compounds.

What is claimed is:

1. Items with an activated surface for immobilisation of macromolecules, including
a substrate with a surface
a dendrimeric scaffold attached to the substrate surface
a number of residues for immobilisation of macromolecules which are linked to the dendrimeric scaffold

2. Items as in claim 1., characterized as:
a sensor or a sensor component,
an array, such as DNA-(micro-)array, a protein-(micro-)array,
a test-array carrying peptides, peptoides or compounds of low molecular weight, like pharmacophors,
a component of a (micro-) reaction vessel or
an optically or electronically active element.

3. Items as in previously mentioned claims, whereby the dendrimeric scaffold includes a number of dendrimeric basic units which have been chosen from the following group: organic dendrimers, starburst-dendrimers, metal-dendrimers, semi-metal dendrimers, carbosilane dendrimers, polysilane dendrimers, glycosyl-containing dendrimers, saccaride- and oligosaccharide-dendrimers, peptide- and oligopeptide-dendrimers, nucleotide- and oligonucleotide-dendrimers as well as derivates of the above mentioned dendrimers.

4. Items as in previously mentioned claims, whereby the dendrimeric scaffold contains dendrimeric basic units which are cross-linked to each other.

5. Procedure for the production of an item with activated surface for immobilisation of macromolecules, with the following steps:
provision of a substrate with a surface
linkage of the substrate surface with dendrimeric basic units and
supply of dendrimeric basic units with a number of primary residues for immobilisation of macromolecules.

6. Procedures according to claim 5, whereby substrate surfaces are supplied with a number of initial residues for an attachment of dendrimeric basic units.

7. Procedures according to claim 6, whereby initial residues were chosen from the following group: hydroxyl-, amino-, carboxyl-, ester-, acylhalogenid-, aldehyde-, epoxy- and thiol-residues as well as disulfides, metalchelats, nucleotides and oligonucleotides, peptides and haptenes, such as biotin-, digoxigenin-, dinitrophenyl-residues.

8. Procedures according to one of claims 5 to 7, whereby the substrate contains a carrier material which was chosen from the following group: carrier materials based on metals, semi-metals, semi-conductive materials, metal-, semi-metal- and non-metal-oxides; glass; plastics; organic and inorganic polymers; organic and inorganic films; gels and monolayers, especially as coating of the above mentioned materials.

9. Procedures according to one of claims 5 to 8, whereby dendrimeric basic units were chosen from the following group: Starburst-dendrimers, metal-dendrimers, glycosyl-containing dendrimers, peptide- and oligopeptide-dendrimers, nucleotide- and oligonucleotide-dendrimers as well as derivates of the above mentioned dendrimers.

10. Procedures according to one of claims 5 to 9, whereby dendrimeric basic units are provided with a number of primary residues for immobilisation of macromolecules by reaction of a functional residue of the dendrimeric basic units with a bifunctional linker substance.

11. Procedures according to claim 10, whereby dendrimeric basic units are supplied with primary residues prior an attachment of the dendrimeric basic units to the substrate surface.

12. Procedures according to claim 10, whereby dendrimeric basic units are supplied with primary residues after an attachment of the dendrimeric basic units to the substrate surface.

13. Procedures according to one of claims 5 to 12, whereby dendrimeric basic units are cross-linked to form a dendrimeric scaffold.

14. Procedure for the production of an item with an immobilised macromolecule on the surface, using following steps:
Contact of a macromolecule containing at least a second residue
(a) with an item according one of the claims 1 to 4 or
(b) with an item containing an activated surface which was produced according to claims 5 to 13
under conditions where at least the first residue of the item and a second residue of the macromolecule chemically react to form a linkage between macromolecule and item.

15. Procedures according to one of claims 5 to 14, whereby the macromolecule and macromolecules, respectively were chosen from the following group: bioorganic macromolecules such as nucleic acids, antibodies, enzymes, receptors, membrane proteins, glycoproteins, carbohydrates, macromolecular and colloid nanoparticles, compounds with low molecular weight, such as pharmacologically active substances, hormones, antigens.

16. All obtainable items, according to claims 6-15, characterised by the procedure they were produced with.
